(19) United States
(12) Patent Application Publication
(10) Pub. No.: US 2007/0276457 A1
(43) Pub. Date: Nov. 29, 2007

(54) Infrared heating devices & therapy for chronic lower back pain

(76) Inventor: Lawrence Gordon, Acton (CA)

Correspondence Address: OSHA LIANG L.L.P., 1221 MCKINNEY STREET, SUITE 2800, HOUSTON, TX, 77010, US

(21) Appl. No.: 11/444,563

(22) Filed: May 31, 2006

Related U.S. Application Data
(60) Provisional application No. 60/809,051, filed on May 26, 2006.

(51) Int. Cl. A61F 7/00; A61F 7/12; A61F 5/00

(52) U.S. Cl. 607/96; 602/14

(57) ABSTRACT

An infrared heating device and therapy for lower back pain includes a belt fastenable around a user so that the belt is overlaying at least a portion of a lumbar region of the user. An infrared emitting element is adjustably positionable between the belt and at a selected area of the user's lumbar region where a user desires pain relief therapy. A portable power supply is attachable to the belt and electrically connectable to the infrared emitting element so that the user can receive infrared therapy at specific selected areas of the lower back while moving, standing, sitting, reclining or sleeping.

### RELATED APPLICATIONS

The present application claims the benefit of priority to co-pending and co-owned provisional application titled INFRARED HEATING DEVICES & THERAPY FOR CHRONIC LOWER BACK PAIN, filed by the present applicant on May 26, 2006, as application Ser. No. ______ (serial number not yet assigned) and such prior provisional application is incorporated herein by reference.

### BACKGROUND OF INVENTION

Chronic lower back pain is one of the most common causes of disability in North American society.

A need for a device for the treatment of back pain and including chronic back pain has been found by the inventor to exist.

### BRIEF DESCRIPTION OF DRAWINGS

FIG. 1 is a perspective view of an infrared heating device according to one embodiment of the invention.

FIG. 2 is a cross-section view of an infrared emitting element for the infrared heating device of FIG. 1 taken along section line 2-2.

FIG. 3 is an exploded assembly view of an infrared emitting element for an infrared lumbar device according to one embodiment of the invention.

FIG. 4 is a perspective view of an infrared emitting element, portable rechargeable power source and re-charger.

FIG. 5 is a schematic depiction of an embodiment of an infrared lumbar device secured around a user and demonstrating a method of use.

FIG. 6 is a schematic depiction of an embodiment of an infrared lumbar device secured around a user and demonstrating a method of use.

### DETAILED DESCRIPTION

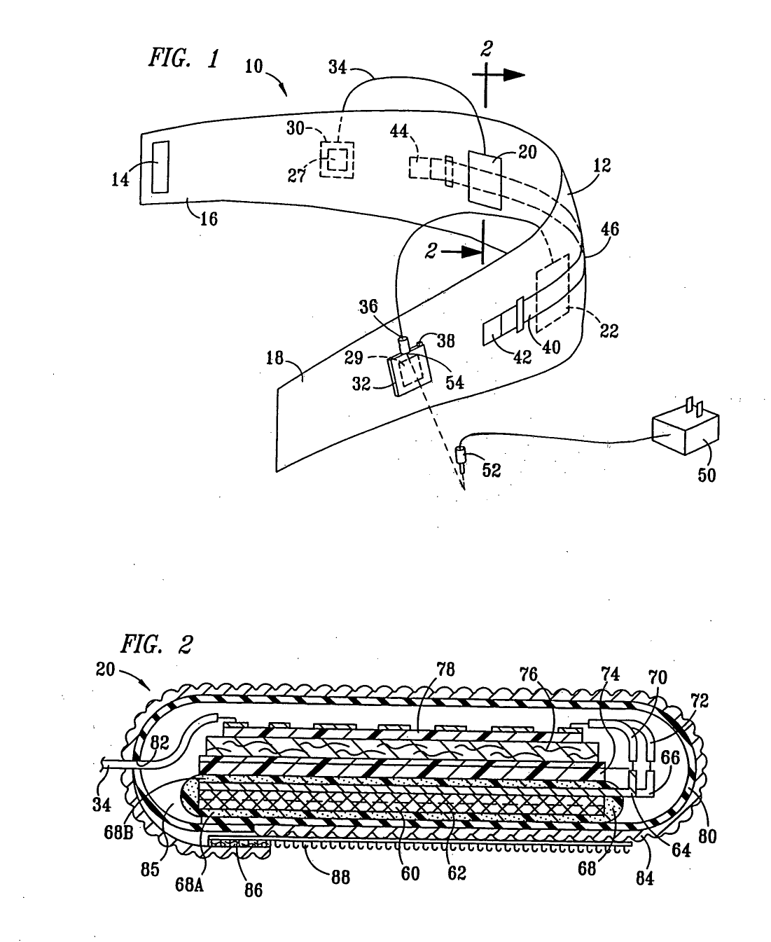

FIG. 1 depicts one embodiment of a therapeutic infrared heating device 10 for use in treating chronic lower back pain. The infrared heating device 10 includes a lumbar belt 12 that may be made from a fabric material capable of being secured, as for example with a fastener 14, around a user so that a portion of the user's lower back or lumbar region that is to be treated is at least partially covered. At least one infrared emitting element 20 is attachable to the lumbar belt 12 at positions corresponding, when the lumbar belt is worn by the user, to locations on a user's body where treatment may be desired. At least one portable power source 30 may also be attached to the lumbar belt 12 at a variety of positions. One or more additional infrared emitting elements 22 may be attachable to the belt. In some embodiments, rather than powering the additional IR emitting element by the same power source, one or more additional power sources 32 may also be attachable for powering the additional infrared emitting element 22 might also be attached t to the lumbar belt 12 as desired by the user. For example the power source 30 may be attachable on the lumbar belt away from the user's body for purposes of comfort.

It has been found by the applicant that a lumbar belt 12 made of a hook-and-loop component material is useful for certain aspect of the invention. The loop component of a hook-and-loop fabric connector system has been found to be useful for any of a variety of purposes, including for purposes of providing comfort to the user because the loop portion is generally softer than the hook portion of the hook-and-loop fastener system, for purposes of securing the belt around the user where the fastening device 14 may include a patch of hook material 14 at one end 16 of the belt 12 so that the loop material at the other end 18 can be secured, and for purposes of convenient attachment of infrared emitting elements 20 and 22 at any desired position on the belt 12 where the belt at least partially overlays a portion of the user's body where therapeutic treatment is desired when the belt 12 is secured around the user's body. One example of a loop portion of the loop fabric material for the belt 12 is known as G96 Medical Laminate that is available from Velcro Company, USA.

In one embodiment, the belt 12 is secured around the user and each IR emitting element is provided with corresponding portion of the hook-and-loop fastening system material. In the case where the belt is loop material, patches of hook material may be secured to any number of infrared emitting elements 20 and 22, and more if desired (additional IR emitting elements are not shown in the drawing for purposes of clarity.) The infrared emitting elements 20 and 22 are sometimes referred to herein as IR emitting elements, IR heating elements, or IR emitters. According to one alternative embodiment the IR emitters can be positioned and temporarily secured at any number of locations for a desired period of time for treatment to be applied and may be repositioned periodically at uniform intervals or at irregular intervals for desired treatment purposes, such as facilitating dissipation of energy or to reduce potential build up of moisture or heat.

In one embodiment each portable energy source 30 or 32 is attachable to the lumbar belt 12 with a patch 27 or 29, respectively, of a hook-and-loop component material. In an embodiment where the lumbar belt is comprised of loop material, a patch of hook material can be secured to the power source. It has been found by the applicant that it is useful to provide for positioning of the power source on the outside surface of the lumbar belt to facilitate user comfort. It is also useful in one alternative embodiment the position for each power source might be changeable, for example so that the user can carry the portable power source on the belt in one position while standing, another position while sitting in a chair and another position for reclining. The changeable position is facilitated by the hook and loop connection and may be further useful for example for purposes of comfortable sleeping whether sleeping on one's back, one one's side or on one's front or simply moving the power source depending upon the user's movement from one position to another for sleeping comfort.

In one alternative embodiment the lumbar belt 12 may be provided with a synching strap 40 to increase curvature or tightness in middle or small of the user's back. In one embodiment the synching strap 40 may be secured in a central region 46, for example with stitching, and tightened, as by using tightening patches 42 and 44 of hook material, on either side independently or on both sides of the secured central region 46. The tightening or loosening can also be useful for increasing or lessening the contact force between one or more IR emitters and the user's body.

In one embodiment the power source 30 or the multiple power sources 30 and 32 may be comprised of one or more replaceable batteries providing the desired voltage to provide appropriate infrared emissions. In one embodiment, the power sources 30 and 32 are provided with electrical connectors 34 and 36, respectively for connecting to the IR emitters 20 and 22 respectively. An on-off switch 38 may be provided to allow the user to control the input of power to the IR emitter. In one embodiment the voltage desired is between about 8 volts and 24 volts. In an alternative embodiment the power source may comprise one or more rechargeable batteries. For example, a re-charger 50 is shown having a plug-in connector 52 adapted to plug in to the power source 30 at receptacle 54. For example, a rechargeable pack of batteries with appropriate voltage, current and power output capabilities has been found by the inventor to be useful. For example NiCad batteries, nickel metal hydride batteries, lithium ion batteries, or lithium polymer (LIPO) batteries might each be useful. The NiCad batteries might be inexpensive, the nickel metal hydride batteries might provide a balance of economy and duration of power, the lithium ion batteries might be light weight and powerful, the LIPO batteries might be useful for traveling and for convenient formability. The inventor has found rechargeable batteries to be useful that have output of about 8 volts to 18 volts at about 500 milliamps to about 1000 milliamps. A type of battery, a size, and/or capacity that will provide power for about 6 to 12 hours before recharging is also useful. The re-charger 50 is selected and/or designed according to known principles for the type of rechargeable battery used.

FIG. 2 shows a section view taken along section line. In the embodiment shown, the infrared emitting element 20 is formed of a carbon and polymer matrix mixture 60 formed on a fiberglass lattice 62. In this embodiment there are spaced apart conductor bars 64 and 66 secured along opposed sides of the matrix mixture 60. The matrix mixture 60 is embedded and surrounds the fiberglass lattice 62 to obtain structural support therefrom. The electrical conductors 64 and 66 are secured in electrical conducting contact with the matrix mixture 60 and the matrix mixture and conductor bars are all encased in Mylar lamination 68 so that only ends of the electrical conductors are exposed for attachment of power leads 70 and 72. The laminated structure 68 is placed adjacent to a plastic support board 74 and an insulation layer 76 is interposed between the support board 74 and a control circuit 78 through which power is transmitted from the connector 34 and the power source 30 (not shown in FIG. 2, see FIGS. 1, 4, 5, and 6) to the conductor bars 64 and 66 and the matrix mixture 60 of the infrared emitting element 20. In one embodiment the control circuit may include temperature control circuitry 79. According to another embodiment the control circuit 78 might also include circuitry to provide one or more other useful control functions for various purposes as would be understood by those of ordinary skill in electrical circuitry arts, such as isolating the components of the infrared device 10 from extraneous power surges, it might regulate the voltage, the current, and/or the total power input to the infrared emitting element 20. A PVC moisture resistant and electrical insulating enclosure 80 is provided surrounding Mylar 68 encased carbon and polymer matrix mixture 60 with spaced apart conductor bars 64 and 66 formed on a fiberglass lattice 64, the support board 74, the insulation layer 76, and the temperature control circuit 78. The moisture resistant PVC enclosure 80 is sealed at 82 to resist penetration of moisture and to avoid inadvertent contact with control circuit 78. A removable cloth moisture absorption and dissipation envelope 84 is also provided surrounding the PVC moisture resistant enclosure. In one embodiment the cloth envelope is formed of terry cloth material to facilitate wicking and absorption of moisture from between the user's body and the infrared emitting element. In an embodiment where the lumbar belt comprises a loop component of hook and loop material, a patch 88 of a hook component of hook and loop material may be secured to the cloth envelope so that selectable attachment of the infrared emitting element to the lumbar belt 12 (see FIGS. 1, 5, and 6) is provided. The cloth envelope 84 may be usefully constructed to have one end 85 that is openable and closable with a flap 86 to allow replacement of one envelope with another while using the same infrared emitting element. For example, when one envelope absorbs moisture it might be replaced with a dry envelope to facilitate comfortable ongoing infrared treatment while the first envelope is allowed to dry or is cleaned and dried for a next use. In one embodiment the cloth forming the envelope both provides wicking of moisture and also includes a component of “loops”, as with a terry cloth material, so that a flap of the same material can be closed onto the same hook material used to removable secure the envelope and its contents (the IR emitter 20) onto the lumbar belt 12.

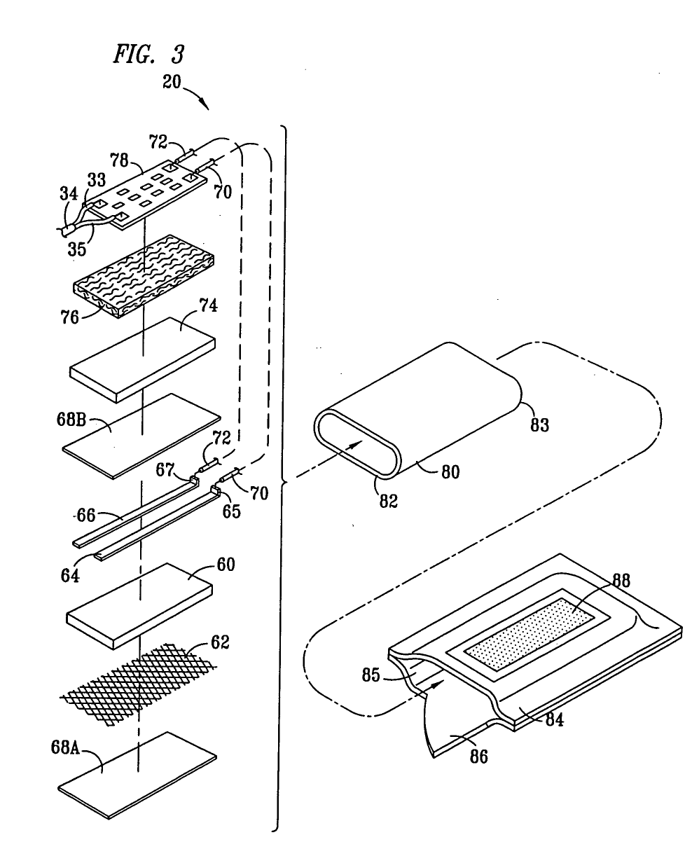

FIG. 3 shows an exploded assembly view of an infrared emitting element 20. In this embodiment the infrared emitting element 20 is shown being constructed of a carbon and polymer matrix mixture 60 formed on a fiberglass lattice 62. In one embodiment the carbon may include graphite and the polymer may include Teflon. It has been considered by the inventor that deeper penetration into the user's body can be accomplished with long wavelength infrared such as infrared in a range above about 700 nm. For example, while infrared of any frequency is considered to be beneficial for treatment and reduction of some kinds of pain in the human body, infrared in a range of about 800 nm to about 1200 nm has been found to be useful for some purposes of the present invention. The proportion of carbon to polymer matrix mixture 60 in the infrared emitting element 20 in combination with the voltage and current used can be adjusted to modify the frequency of the wave length of the infrared emitted from the IR emitter 20. For example wave lengths of about 850 nm might be usefully targeted to be emitted by the selected matrix mixture 60.

In the shown in FIG. 2 there are spaced apart conductor bars 64 and 66 secured along opposed sides of the matrix mixture 60. The matrix mixture 60 is embedded and surrounds the fiberglass lattice 62 to obtain structural support therefrom. The electrical conductors 64 and 66 are secured in electrical conducting contact with the matrix mixture 60 and the matrix mixture and conductor bars are all encased in a material that substantially transparent to infrared wavelength electromagnetic radiation, such as Mylar lamination 68, that may be formed of laminate layers 68A and 68B so that only ends 65 and 66 of the electrical conductors 64 and 66, respectively, are exposed for attachment of power leads 70 and 72. The laminated structure is placed adjacent to a plastic support board 74 and an insulation layer 76 is interposed between the support board 74 and a control circuit 78 through which power is transmitted from the connector 34, that may include wires 33 and 35 communicating the electrical power from the power source 30 (not shown in FIG. 3, see FIGS. 1, 4, 5, and) to the conductor bars 64 and 66 and the matrix mixture 60 of the infrared emitting element 20. It has been found useful to maintain a size of the infrared emitting element relatively small so that localized and contrasting infrared radiation therapy can be applied at selected locations where pain has been experienced by the person using the device. For example, an infrared emitter that is rectangular and has dimensions of between about 2.5 cm and 7.5 cm (about 1 inch and 3 inches) in one direction and between about 5 cm and 15 cm (2 inches and 5 inches) in another direction may be useful for localizing the infrared radiation. The thickness of the infrared element may be relatively thin, for example about 0.5 cm to 1.5 cm, to facilitate comfortable use.

The control circuit 78 is designed to permit transfer of power when the temperature is below selected, desired, or predetermined maximum limits, and to reduce power transfer when the temperature exceeds predetermined limits. For example, for safety and to avoid the sensation of being too hot the maximum temperature might be selected at or below about 40-45° C. (about 105-115° F.), such as below about 42° C. Other temperatures might be selected depending upon the treatment and the user's tolerance to the sensation of elevated temperatures, as for example up to about 70° C. (about 160° F.) that might be tolerated by certain users under carefully controlled circumstances, such as by continuous observation and monitoring by a trained professional during use. A PVC moisture resistant and electrical insulating enclosure 80 is provided surrounding the assembly including the Mylar 68 encased carbon and polymer matrix mixture 60 with spaced apart conductor bars 64 and 66 formed on a fiberglass lattice 64, the support board 74, the insulation layer 76, the control circuit 78, and the electrical conductors 70 and 72 and wires 33 and 35 of power cable 34. The moisture resistant PVC enclosure 80 is sealed at both ends 82 and 83 to resist penetration of moisture and to avoid inadvertent contact with control circuit 78 and the conductors 70 and 72 and wires 33 and 35. A removable cloth moisture absorption and dissipation envelope 84 is also provided surrounding the PVC moisture resistant enclosure 80. In one embodiment the cloth envelope 84 is formed of terry cloth to facilitate wicking of moisture from the user's body between the infrared emitting element 20 and the user's body. The cloth envelope 84 may have an openable end 85 that can receive the infrared emitting element 20 and that is closable with a flap 86 to hold the infrared emitting element inside the envelope 84. A patch 88 of hook and loop component material may be secured to the cloth envelope 84 to allow selectable attachment of the infrared emitting element 20 to the lumbar belt 12 of FIG. 1.

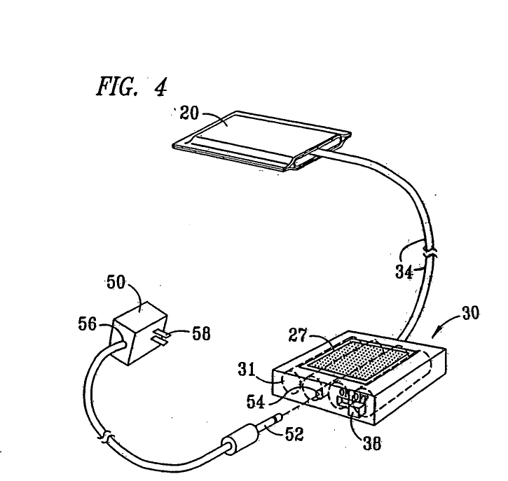

FIG. 4 shows an infrared emitting element 20 connected by a power cable 34 to a portable power source 30. While the power source may be formed using one or more replaceable batteries that generate a desired voltage and current, for example a DC voltage of more than about 8 volts and up to about 24 volts and a current of more than about 500 milliamps, a portable rechargeable power source such as a pack including a plurality of re-chargeable batteries 31, or a plurality of re-chargeable battery cells, has been found to be useful for certain purposes. For example NiCad batteries 31, nickel metal hydride batteries 31, lithium ion batteries 31, or lithium polymer (LIPO) batteries 31 might each be useful for various purposes of constructing a rechargeable and portable power source 30 consistent with various aspects of the invention. For example, NiCad batteries might be inexpensive, nickel metal hydride batteries might provide a balance of economy and duration of power, the lithium ion batteries might be light weight and powerful, the LIPO batteries might be useful for traveling and for convenient formability because they can be conveniently formed having generally flat rectangular dimensions. The inventor has found rechargeable batteries to be useful that have output of about 8 volts to 24 volts at about 500 milliamps to about 1000 milliamps. A type of battery, a size, and/or capacity that will provide power for about 4 to 12 hours or more before recharging is also useful. In one embodiment a voltage of about 8-10 volts at about 700 milliamps might be provided by a NiCad battery having a size sufficiently small to be portable and held by the lumbar belt and sufficiently large to provide power for a period of about 5-6 hours. In another embodiment a voltage of about 12 to 14 volts might be provided at about 800 milliamps by a Lithium Ion battery pack that can power the infrared emitting element for about 6-8 hours. In another embodiment a voltage of about 14-18 volts at about 700-900 milliamps might be provided by a Lithium polymer (LiPO) battery pack constructed to power the infrared heating element for a period of up to about 12 hours. In one embodiment the portable power source 30 is provided with an activator device 37, such as a user operated on-off switch to activate the flow of current from the power source to the power cable 34 and into the infrared emitting element 20 The re-charger 50 is selected and/or designed according to known principles for the type of rechargeable battery used. For example, those of ordinary skill in the portable battery arts will understand base upon the present disclosure that a re-charger 50 capable of recharging at a voltage slightly higher than the desired battery delivery voltage may be appropriate depending upon the type of battery selected. The re-charger 50 may include a receptacle 54 formed and connecting into the power source 30 that can receive a connector pin 52 that is coupled to a re-charger circuit 58 that converts standard household current received at prongs 58 into recharging current.

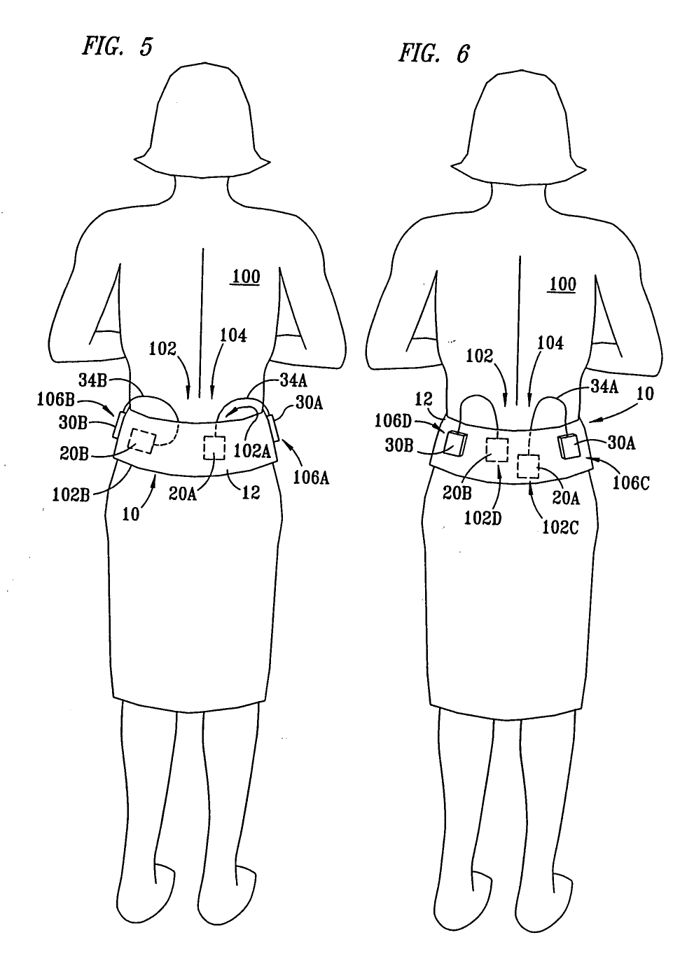

FIG. 5 is a schematic depiction of an embodiment of a method of using an infrared lumbar device 10 so that a lumbar belt 12 is secured around a user's body 100 at least partially covering a portion 102 of the lower back 104 or lumbar region 104 of the user's body 100 where infrared therapy is desired. According to one embodiment of a method for infrared treatment of chronic lower back pain, an infrared emitting element 20A is positioned and secured to the lumbar belt 12 and secured via hook and loop faster components between the belt 12 and the body 100 a location 102A to be treated. Fore example at a location 102A of the lower back region 104 on the user's body 100 where lower back pain is experienced by the user periodically or chronically. A portable power source 30A is electrically connected to the infrared emitting element by a power cable 34A. The portable power source 30A may be moveably secured to the belt 12 at a convenient location 106A for easy access or otherwise for the convenience or comfort of the user. For example, the user might select any number of locations such as in the front or at the side for use while walking, sitting, or reclining or sleeping on the user's back.

One or more additional infrared emitting elements 20B might also be positioned at one or more other locations 102B where treatment is desired, such as other specific locations 102B toward a side of the lower back region 104 on the user's body 100 where lower back pain is experienced by the user. A portable power source 30B is electrically connected to the infrared emitting element by a power cable 34B. The portable power source 30B may be moveably secured to the belt 12 at a convenient location 106B for easy access or otherwise for the convenience or comfort of the user.

FIG. 6 is a schematic depiction of an embodiment of a method of using an infrared lumbar device 10 so that a lumbar belt 12 is secured around a user's body 100 at least partially covering a portion 102 of the lower back 104 or lumbar region 104 of the user's body 100 at alternative locations where infrared therapy is desired. An infrared emitting element 20A is positioned and secured to the lumbar belt 12 and secured via hook and loop faster components between the belt 12 and the body 100 a location 102C to be treated that is varied slightly from the location 102A of FIG. 5. For example, after a period of use at a location 102A (see FIG. 5) the perceived focus of remaining discomfort or pain sensation might be slightly different such as lower on the lower back region 104 on the user's body 100. A portable power source 30A that is electrically connected to the infrared emitting element 20A by a power cable 34A may be kept in the same place as before or it may be repositioned to another location 106C for the convenience or comfort of the user. For example, the user might select any number of locations such as at the back for use while walking without hindering side movement of the arms, or for sleeping on the user's side or front.

One or more additional infrared emitting elements 20B might also be positioned at one or more other locations 102D where treatment is desired, such as other specific locations 102D toward the middle of the lower back region 104 on the user's body 100 where lower back pain is experienced by the user. The portable power source 30B that is electrically connected to the infrared emitting element 20B by a power cable 34B may be held in the same position as before or may be moveably secured to the belt 12 at another convenient 106D for the convenience or comfort of the user.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A portable therapeutic lumbar device comprising:
a belt fastenable around a user so that the belt is overlaying at least a portion of a lumbar region of a user;
at least one infrared emitting element adjustably positionable between the belt and at a selected area of the user's lumbar region where a user desires pain relief therapy;
a portable power supply attachable to the belt and electrically connectable to the infrared emitting element.

2. The portable therapeutic lumbar device of claim 1, comprising:
a hook and loop connector component attached to the infrared emitting element, and
wherein the belt comprises a hook and loop fabric component for removable attachment to the hook and loop component attached to the infrared emitting element for holding the infrared emitting element at the selected position between the belt and the user's lumbar region.

3. The portable therapeutic lumbar device of claim 1, wherein
the infrared emitting element, comprises a carbon and polymer matrix mixture with spaced apart conductor bars formed on a fiberglass lattice all encased in a substantially IR transparent material.

4. The portable therapeutic lumbar device of claim 3, wherein
the infrared emitting element further comprises support board an insulation layer and a control circuit capable of limiting the temperature of the infrared emitting element.

5. The portable therapeutic lumbar device of claim 4, wherein the infrared emitting element, further comprises:
a PVC moisture resistant enclosure surrounding the carbon and polymer matrix mixture with spaced apart conductor bars formed on a fiberglass lattice, the substantially IR transparent encasement, the support board, the insulation layer, and the control circuit; and
a cloth moisture absorption and dissipation envelope surrounding the PVC moisture resistant enclosure.

6. The portable therapeutic lumbar device of claim 1, wherein:
the portable power supply comprises a patch of hook and loop component and
wherein the belt comprises a hook and loop fabric component for removable attachment to the hook and loop component attached to the portable power supply for holding the portable power supply at the selected position so that the battery pack can be selectably placed on the lumbar belt at a variety of positions for the convenience or comfort of the user.

7. The portable therapeutic lumbar device of claim 1, wherein:
the portable power supply comprises a replaceable battery pack.

8. The portable therapeutic lumbar device of claim 1, wherein:
the portable power supply comprises a rechargeable battery pack.

9. The portable therapeutic lumbar device of claim 8, wherein:
the rechargeable battery pack comprises one or more rechargeable nickel metal hydride batteries.

10. The portable therapeutic lumbar device of claim 8, wherein:
the rechargeable battery pack comprises one or more rechargeable Lithium Ion batteries.

11. The portable therapeutic lumbar device of claim 8, wherein:
the rechargeable battery pack comprises one or more rechargeable Lithium polymer batteries.

12. The portable therapeutic lumbar device of claim 1, wherein:
the at least one infrared emitting element comprises a control circuit capable of reducing the power to the infrared emitting element when a predetermined temperature is exceeded.

13. The portable therapeutic lumbar device of claim 1, wherein:
the at least one infrared emitting element is substantially flat and has a relatively small size with a maximum dimension of about 10 cm and a thickness of about 1 cm.

14. The portable therapeutic lumbar device of claim 1, wherein:
the at least one infrared emitting element comprises a matrix mixture of carbon and polymer.

15. The portable therapeutic lumbar device of claim 13, wherein:
the at least one infrared emitting element comprises a matrix mixture of graphite and Teflon.

16. The portable therapeutic lumbar device of claim 1, wherein:
the at least one infrared emitting element comprises a plurality of infrared emitting elements each selectably positionable on the lumbar belt at a plurality of locations where IR therapy is desired by the user.

17. The portable therapeutic lumbar device of claim 1, wherein:
the at least one infrared emitting element comprises a matrix mixture of carbon and polymer capable of emitting infrared radiation having a wave length of between about 700 nm and 1200 nm

18. A portable chronic back pain therapy device comprising:
a belt formed of a loop component of hook and loop material and fastenable around a user so that the belt is overlaying at least a portion of a lumbar region of a user;
at least one infrared emitting element having a hook portion of hook and loop material attached so that the infrared emitting element is adjustably positionable between the belt and the user's lumbar region at more than one selected areas of the user's lumbar region where a user desires pain relief therapy, the infrared emitting element capable emitting infrared radiation having a wavelength of between about 800 nm and 1200 nm, and
at least one portable power supply having a hook portion of hook and loop material attached so that the portable power supply is removably attachable to the belt at selected locations and electrically connectable to the infrared emitting element for providing a electrical power to the infrared emitting element so that pain relief infrared therapy is applied to the user while undertaking normal activities.

19. A method of treating lower back pain comprising:
covering at least a portion of the a person's lower back with a lumbar belt;
positioning an infrared emitting element between the belt and the lower back at a desired location corresponding to a location where back pain is experienced;
connecting a portable power source to the infrared emitting element so that infrared radiation is emitted and attaching the portable power source to the belt at a location that is convenient and comfortable for the person so that the infrared radiation can be continued while the lumbar belt holds the infrared emitting element in position while normal activity by the person continues.

20. The method of treating lower back pain of claim 19, wherein:
covering at least a portion of a persons lower back with a lumbar belt comprises attaching a belt formed of a loop component of hook and loop material around the waist and lower back of the person, and
positioning an infrared emitting element between the belt and the lower back at a desired location corresponding to a location where back pain is experienced comprises detachably connecting an infrared emitting element to the inside of the lumbar belt with a hook component of hook and loop material so that the infrared emitting element is in contact with user's body at a location where back pain is experienced.
